(19) United States
(12) Patent Application Publication
(10) Pub. No.: US 2008/0221448 A1
(43) Pub. Date: Sep. 11, 2008

(54) Image-guided delivery of therapeutic tools duing minimally invasive surgeries and interventions

(76) Inventors: Butrus T. Khuri-Yakub, Palo Alto, CA (US); Omer Oralkan, Santa Clara, CA (US); Goksen G. Yaralioglu, Mountain View, CA (US)

Correspondence Address: LUMEN PATENT FIRM, INC., 2345 YALE STREET, SECOND FLOOR, PALO ALTO, CA, 94306, US

(21) Appl. No.: 12/072,906

(22) Filed: Feb. 27, 2008

Related U.S. Application Data
(60) Provisional application No. 60/906,097, filed on Mar. 7, 2007.

(51) Int. Cl. A61B 8/00; A61B 18/20; A61N 7/00

(52) U.S. Cl. 600/439

(57) ABSTRACT

Imaged-guided therapy for minimally invasive surgeries and interventions is provided. An image-guided device includes an elongate tubular member, such as a catheter, an annular array of capacitive micromachined ultrasound transducers (cMUTs) for real-time three-dimensional forward-looking acoustic imaging, and a therapeutic tool. The therapeutic tool is positioned inside an inner lumen of the elongate tubular member and can be a device for tissue ablation, such as a high intensity focused ultrasound (HIFU) device or a laser. The HIFU device is operable at high frequencies to have a sufficiently small focus spot, thus a high focal intensity. The imaging annular array is also operable at high frequencies for good acoustic imaging resolution. The high resolution forward-looking imaging array, in combination with the high frequency HIFU transducer, provides a single image-guided therapy device for precise tissue ablation and real-time imaging feedback.

### CROSS-REFERENCE TO RELATED APPLICATIONS

This application claims priority from U.S. Provisional Patent Application 60/906,097 filed Mar. 7, 2007, which is incorporated herein by reference.

### STATEMENT OF GOVERNMENT SPONSORED SUPPORT

This invention was made with Government support under contract NIH awarded by 1-RO1-HL7647 and grant number GPEDC0013B from OHSU. The Government has certain rights in the invention.

### FIELD OF THE INVENTION

The invention relates generally to medical devices. More particularly, the present invention relates to ultrasound image-guided delivery of therapeutic tools for minimally invasive medical procedures.

### BACKGROUND

Minimally invasive techniques are widely used in medical procedures including cardiac, vascular, joint, abdominal, and spinal surgeries and interventions. In minimally invasive interventions, a surgical or medical tool is introduced into the body through a natural body opening or small artificial incisions. A separate endoscopic camera is typically used to obtain optical images inside of the body to help perform the minimally invasive procedure. Minimally invasive medical techniques have several advantages over open surgeries, such as minimizing incision size and trauma, and reducing recovery time. However, existing minimally invasive techniques suffer from restricted vision. Particularly, for cardiac or vascular interventions, blood poses difficulties for optical imaging.

Under conditions when optical imaging is inadequate, ultrasound imaging can be used. Existing intravascular or endoscopic ultrasound imaging devices, however, typically only provide side-looking cross-sectional images. For inserting catheters or other medical devices 20 into a body, side-looking images are inadequate if there is blockage along the direction of insertion. For example, in coronary catheterization, totally occluded or heavily stenosed vessels make it impossible to introduce catheters with strictly side-looking capabilities.

Many existing minimally invasive medical instruments are also typically limited to image only or therapy only capabilities. With separate instruments for imaging and therapy, a medical interventionist would have to either separately introduce the imaging and therapy instruments or introduce multiple catheters or tubes. In the former scenario, the accuracy and guidance capability would be limited and in the latter scenario, the size of the openings must be large enough to accommodate multiple tubes.

Recently, high intensity focused ultrasound (HIFU) techniques have been developed for medical procedures, such as tissue destruction. Traditional HIFU techniques rely on cavitation effects or thermal effects as mechanisms for tissue destruction. Low frequency HIFU is naturally preferred to induce cavitation. Even when thermal effects are desired to be the dominant mechanism of tissue destruction, low frequencies are still preferred because of the increased attenuation at higher frequencies. For at least these reasons, existing HIFU devices for medical procedures typically operate at low frequencies. However, existing low frequency HIFU devices typically do not offer sufficient focal intensity for tissue ablation, especially for HIFU transducers with small diameters. Imaging ultrasounds also typically operate at low frequencies due to the increase in attenuation at higher frequencies.

The present invention addresses the problem of imaging and applying therapy in minimally invasive interventions.

### SUMMARY OF THE INVENTION

The present invention is directed to image-guided therapy using an imaging ultrasound array and a therapeutic tool positioned on the same instrument. The image-guided therapy device includes an elongate tubular member, such as an intravascular or intracardiac catheter, having an inner lumen. The elongate tubular member is dimensioned to fit inside a body lumen. An annular ultrasound array, having a central lumen formed by the annulus of the array, is positioned on a distal end of the elongate tubular member such that the central lumen of the annular ultrasound array is at least partially aligned with the inner lumen of the elongate tubular member. The elements of the annular ultrasound array include multiple capacitive micromachined ultrasonic transducers (cMUTs). The annular ultrasound array is capable of real-time forward-looking imaging. Importantly, the image-guided therapy device also includes a therapeutic tool positioned on the distal end and inside of the inner lumen of the elongate tubular member.

Minimally invasive interventions can be performed by inserting the image-guided therapy device into a body lumen or cavity, imaging a region inside the body lumen by using the annular ultrasound array, guiding the therapeutic tool to a focus spot based on the imaging, and applying therapy to the focus spot by using the therapeutic tool. The interventions can also include scanning over an area inside the body lumen or delivering an ultrasound contrast agent through the inner lumen of the elongate tubular member to enhance the imaging.

In a preferred embodiment, the cMUTs of the imaging annular ultrasound array are operable at a high frequency that is equal to or greater than about 10 MHz. Preferably, the therapeutic tool is a HIFU device, also operable at a high frequency that is equal to or greater than about 10 MHz. The HIFU device can have a diameter of about 2 mm and a focal distance of about 2 mm. The HIFU device can include a single focused transducer element or a phased array transducer. The elements of the phased array can include multiple HIFU cMUTs.

The therapeutic tool can also include medical instruments in replacement of or in addition to the HIFU device, such as a laser for tissue ablation, a device for radio-frequency ablation, a biopsy needle, an atherectomy device, or any other surgical tool. When used in combination with the HIFU device, a biopsy tool can be used to determine the efficacy of the HIFU device. An optical fiber can also be used to perform optical imaging. Correlations of the optical and acoustic imaging can be used to determine the efficacy of a therapeutic tool, particularly a HIFU device.

### BRIEF DESCRIPTION OF THE FIGURES

The present invention together with its objectives and advantages will be understood by reading the following description in conjunction with the drawings, in which:

FIG. 1 shows an example of an image-guided therapy device inserted inside a body lumen according to the present invention.

FIG. 2A shows an example of an image-guided therapy device with a single HIFU transducer according to the present invention.

FIG. 2B shows a cross-sectional view of the image-guided therapy device of FIG. 2A.

FIG. 3A shows an example of an image-guided therapy device with a HIFU transducer array according to the present invention.

FIG. 3B shows a cross-sectional view of the image-guided therapy device of FIG. 3A.

FIG. 4A shows an example of an image-guided therapy device with a laser according to the present invention.

FIG. 4B shows a cross-sectional view of the image-guided therapy device of FIG. 4A.

FIG. 5 shows an example of an image-guided therapy device including a HIFU device, a biopsy tool, and an optical fiber for optical imaging according to the present invention.

### DETAILED DESCRIPTION OF THE INVENTION

Minimally invasive surgeries and interventions require delivery of a therapeutic tool through natural body openings or small artificial incisions. However, many instruments and methods to conduct these interventions suffer from restricted vision. Below is a detailed description of methods and devices for image-guided therapy delivery usable in minimally invasive surgeries and interventions.

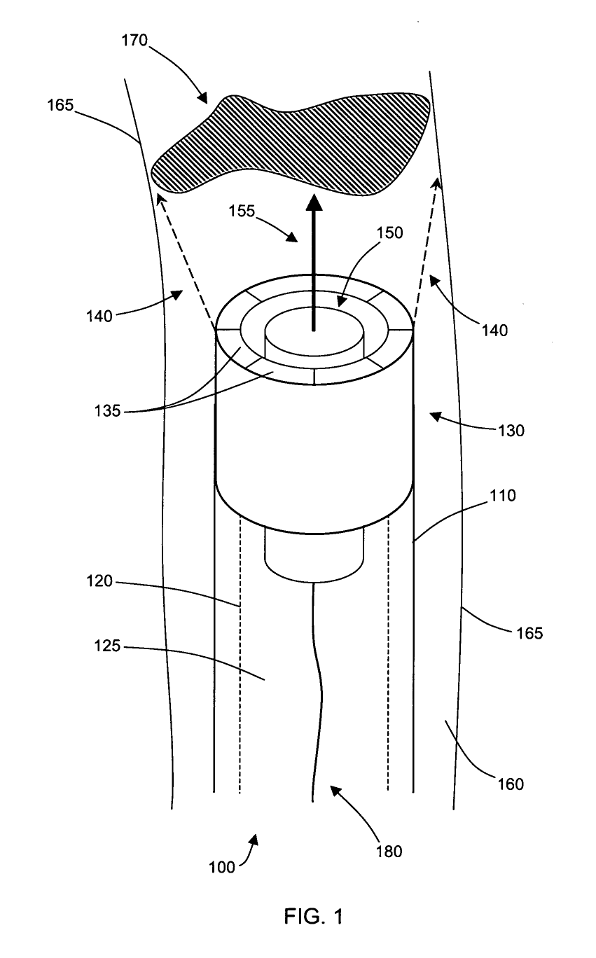

FIG. 1 shows an example of an image-guided therapy device 100 that has been inserted inside of a body lumen 160. The image-guided therapy device 100 includes an elongate member, such as a catheter or an endoscopic instrument, dimensioned to fit inside of the body lumen 160. The elongate member is tubular and has an outer wall 110 and an inner wall 120. The inner wall 120 forms the inner lumen 125 of the elongate tubular member.

Located at the distal end of the elongate tubular member are the acoustic imaging and therapy components of the image-guided therapy device 100. An annular ultrasound array 130 is positioned on the distal end for real-time forward-looking imaging. The annulus of the annular ultrasound array 130 defines a central lumen, wherein the central lumen of the annular ultrasound array 130 and the inner lumen 125 of the elongate tubular member are at least partially aligned. Positioned inside of the inner lumen 125 is a therapeutic tool 150. Optionally, electronic components and cabling 180 can also be placed inside the inner lumen 125 or between the inner wall 120 and the outer wall 110.

As shown by FIG. 1, the image-guided therapy device 100 has been inserted into a body lumen 160. The body lumen 160 can be a natural body lumen, such as a blood vessel, with body lumen walls 165, or the body lumen 160 can be created by an artificial incision. The body lumen 160 can also represent any body cavity or region of the human or animal body, and the image-guided therapy device 100 can be positioned to that region through a natural body opening or through an incision. The device 100 can be moved to an inner body region 170 where a therapeutic procedure is required or desired. Using the annular ultrasound array 130, a part of the inner body region 170 can be imaged to accurately guide the therapeutic tool 150 to a focus spot. The therapeutic tool 150 can then be used to apply therapy to the focus spot.

It is important to note that the elements 135 of the annular ultrasound array 130 are preferably capacitive micromachined ultrasound transducers (cMUTs). The annular ultrasound array 130 can contain any number of elements. Using an array of cMUTs, the image-guided therapy device 100 has three-dimensional forward-looking capabilities. The dashed lines with arrows 140 in FIG. 1 schematically indicate the field of view of the image-guided therapy device 100. Unlike conventional side-looking devices, forward-looking imaging enables a medical interventionist to not only guide the image-guided therapy device 100 to the location for treatment, but also to provide real-time feedback during the therapeutic procedure.

Furthermore, the combination of high resolution imaging and real-time feedback enables a medical interventionist operating the device 100 to precisely position the therapeutic tool 150. Additionally, with a forward-looking annular ultrasound array 130 and a therapeutic tool 150 on the same device 100, the medical interventionist can scan an area of the inner body region 170, where scanning involves imaging the area and applying therapy to some or all of the area. With separate devices for imaging and therapy, an interventionist would be required to repeatedly remove and introduce multiple separate devices or introduce multiple catheters into the same body lumen.

In a preferred embodiment, the therapeutic tool 150 includes a high intensity focused ultrasound (HIFU) device. The HIFU device can operate at a high frequency equal to or greater than about 10 MHz. In HIFU devices, focusing gain increases with frequency. More particularly, the size of the focus spot of the HIFU device is related to the acoustic wavelength (λ) and the focal distance (z), defined as the distance from the HIFU device to the focus spot. The focus spot size is also inversely related to the diameter (d) of the HIFU device. These qualitative relations show that a decrease in λ (or, equivalently, an increase in frequency) would decrease the focus spot size. By decreasing the size of the focus spot, the focusing gain increases, thereby producing a large focal intensity necessary for certain therapeutic applications, such as tissue ablation or coagulative necrosis. Furthermore, a smaller focus spot may be desired for precise application of therapeutic acoustic energy.

Increasing the diameter of the HIFU device can also reduce the size of the focus spot and increase the total available power. Many minimally invasive interventions, such as intravascular or intracardiac procedures, however, require small devices to fit inside certain body lumens. The diameter of the HIFU device is limited in these restrictive environments as the size of the body lumen constrains device dimensions. The small size and large focal intensity requirements can be overcome by operating a small diameter HIFU device at high frequency. At high frequencies, the focus spot size can be greatly reduced giving the required large focal intensity. However, increasing the frequency of the HIFU device also increases attenuation. The competition between a decrease in spot size (i.e. an increase in focusing gain) and an increase in attenuation introduces an optimal frequency. The optimal frequency can be measured or found based on calculations or simulations.

In fact, it is found that a 2 mm diameter HIFU device operating at its optimal frequency of 60 MHz results in a focal intensity of about 8 kW/cm² at a focus spot with a diameter of about 0.025 mm at a focal distance of about 2 mm. This focal intensity is comparable to the intensity from a 50 mm diameter HIFU operating at 2.5 MHz, resulting in a focus spot with a diameter of about 0.6 mm at a focal distance of about 50 mm. In a preferred embodiment, the high frequency HIFU device has a diameter of about 2 mm and a focal distance of about 2 mm. When operated at about 10 MHz, the focus spot diameter of the preferred HIFU device is about 0.15 mm. Alternatively, other dimensions of the HIFU device and operating frequencies can be used.

It is important to note that the cMUTs of the annular ultrasound imaging array 130 are operable at high frequency, including frequencies equal to or greater than about 10 MHz. High frequency imaging increases the resolution of the annular ultrasound array 130. The high resolution may be necessary due to the small focus spot from the high frequency HIFU device. Optionally, to enhance the quality of the acoustic imaging, one or more ultrasound contrast agents can be introduced to the inner body region 170. The ultrasound contrast agent can be delivered through the inner lumen 125 of the elongate tubular member.

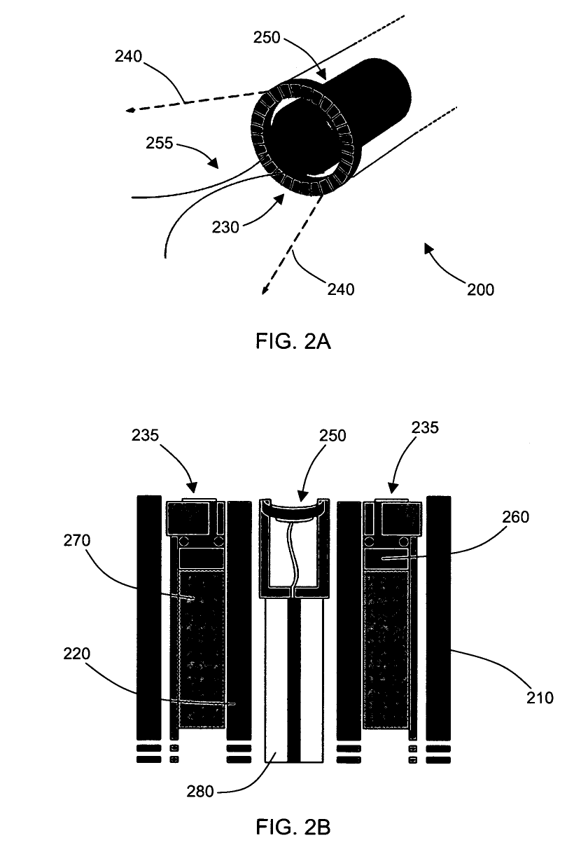

FIG. 2A shows an exemplary image-guided therapy device 200 with a single HIFU transducer 250 for providing HIFU therapy at regions proximate to a focus spot 255. The single HIFU transducer 250 can be a cMUT or a piezoelectric transducer. FIG. 2B shows a cross-sectional view of the device in FIG. 2A. The inner lumen formed by the inner wall 220 contains the single transducer 250 and other housing 280 for electrical components and cabling. FIG. 2B also shows support structures 270 and electronics 260 between the inner wall 220 and outer wall 210. The support structures 270 can be made of alumina and the electronics 260 can be for operating the imaging cMUT elements 235 of the annular ultrasound array 230.

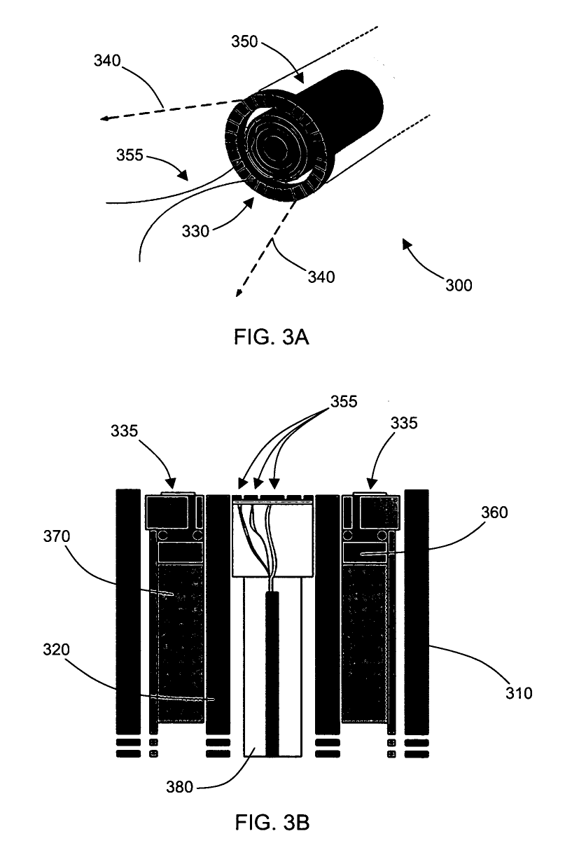

FIG. 3A and FIG. 3B show another exemplary image-guided therapy device 300 similar to the device shown in FIG. 2A and FIG. 2B. However, the single HIFU transducer 250 is replaced by a phased array 330 of therapeutic HIFU cMUT elements 355. The HIFU phased array 330 is focused electronically by providing phased excitation signals to different elements 355 of the array 330. FIG. 3A and FIG. 3B show a HIFU phased array 330 configured as concentric annular rings, however any phased array configuration can be employed.

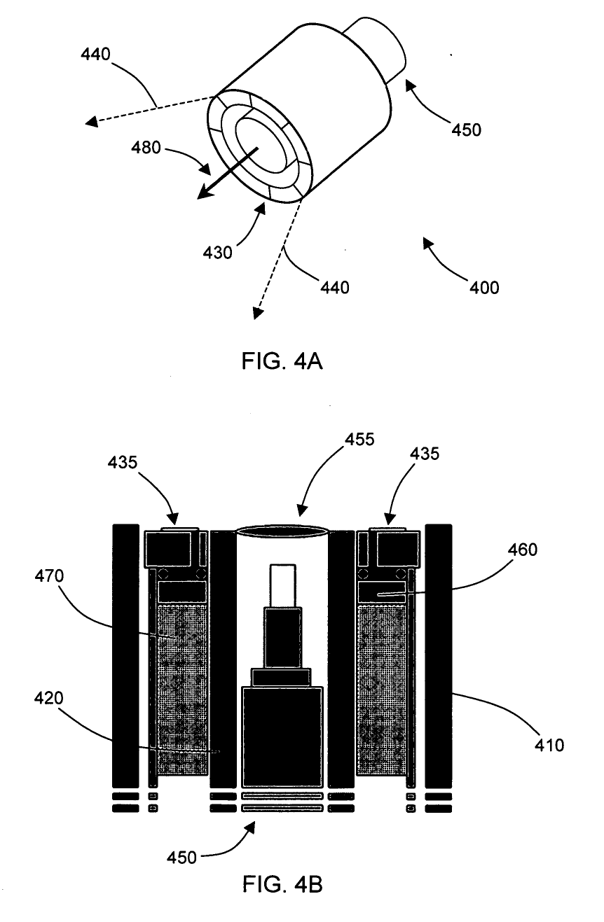

In addition to a HIFU device, the therapeutic tool 130 of the image-guided therapy device 100 can include other surgical tools, such as a laser for tissue ablation, a device for radio-frequency ablation, a biopsy needle, an atherectomy device, or any other surgical tool. FIG. 4A shows another exemplary image-guided therapy device 400 with a laser 450. FIG. 4B shows a cross-sectional view of the device 400 of FIG. 4A. The laser 450 is focused with a lens 455 to form a laser beam 480 for tissue ablation. Components for the laser 450 can be housed in the inner lumen of the device 400.

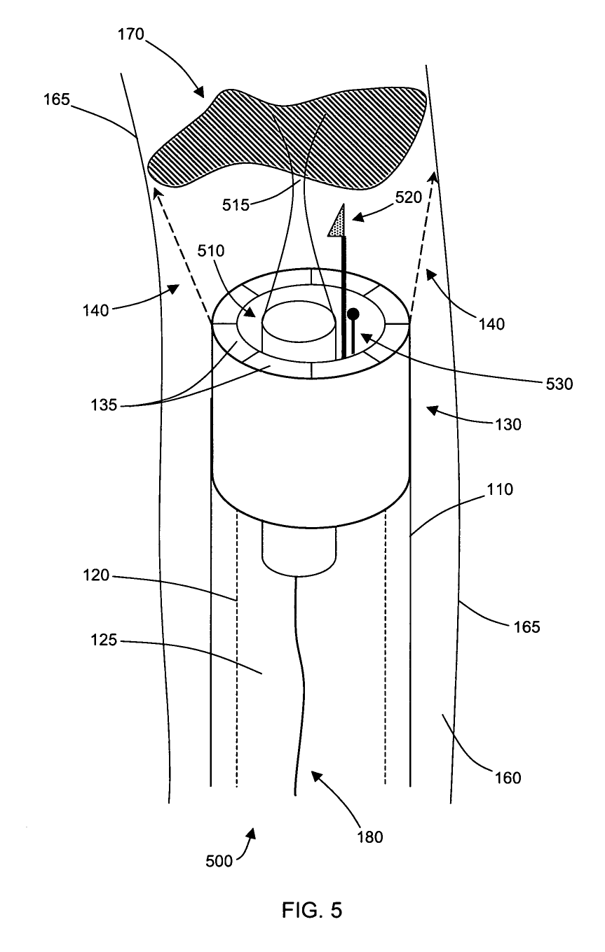

FIG. 5 shows an embodiment of an image-guided therapy device 500 including a HIFU device 510, a biopsy tool 520, and an optical fiber 530, all of which are positioned inside the inner lumen 125 of the device 500. The biopsy tool 520 can be used to extract tissue from a region 170 inside the body lumen 160. The extracted tissue can be analyzed to determine the efficacy of the therapy from the HIFU device 510. Similarly, the optical fiber 530 can be used to determine the efficacy of the therapy from the HIFU device 510. The optical fiber 530 can perform optical imaging, where the optical images and the acoustic images from the annular imaging ultrasound array 130 can be correlated for the efficacy determination. Additional sensory devices, such as electrophysiology sensors or pressure sensors, can also be placed in addition to or replacement of the therapeutic tool inside the inner lumen 125 of the elongate tubular member.

As one of ordinary skill in the art will appreciate, various changes, substitutions, and alterations could be made or otherwise implemented without departing from the principles of the present invention, e.g. other surgical tools can be positioned inside the inner lumen and the imaging ultrasound array can be configured in any geometry. Accordingly, the scope of the invention should be determined by the following claims and their legal equivalents.

What is claimed is:

1. An image-guided therapy device, comprising:
(a) an elongate tubular member having a distal end and an inner lumen, wherein said elongate tubular member is dimensioned to fit inside a body lumen;
(b) an annular ultrasound array for real-time forward-looking acoustic imaging, wherein said annular ultrasound array is positioned on said distal end of said elongate tubular member, wherein said annulus of said annular ultrasound array defines a central lumen of said annular ultrasound array, wherein said central lumen of said annular ultrasound array and said inner lumen of said elongate tubular member are at least partially aligned, and wherein said annular ultrasound array comprises a plurality of capacitive micromachined ultrasonic transducers (cMUTs); and
(c) a therapeutic tool, wherein said therapeutic tool is positioned near said distal end of said elongate tubular member and is positioned inside said inner lumen of said elongate tubular member.

2. The device as set forth in claim 1, wherein said cMUTs of said annular ultrasound array are operable at a high frequency, and wherein said high frequency is equal to or greater than about 10 MHz.

3. The device as set forth in claim 1, wherein said therapeutic tool comprises a high intensity focused ultrasound (HIFU) device, wherein said HIFU device is operable at a high frequency, and wherein said high frequency is equal to or greater than about 10 MHz.

4. The device as set forth in claim 3, wherein said HIFU device has a diameter of about 2 mm, wherein said HIFU device focuses ultrasonic energy to a focus spot, wherein said focus spot is located about 2 mm away from said HIFU device, and wherein the size of said focus spot is dependent on the frequency of said HIFU device.

5. The device as set forth in claim 3, wherein said HIFU device comprises a single focused transducer element.

6. The device as set forth in claim 3, wherein said HIFU device comprises a phased array of a plurality of HIFU cMUTs.

7. The device as set forth in claim 3, further comprising a biopsy tool, wherein said biopsy tool is positioned inside said inner lumen of said elongate tubular member, wherein said biopsy tool is for extracting tissue from inside said body lumen, and wherein said extracted tissue is usable to determine an efficacy of the therapy from said HIFU device.

8. The device as set forth in claim 3, further comprising an optical fiber for optical imaging, wherein said optical imaging and said acoustic imaging can be correlated to determine an efficacy of the therapy from said HIFU device.

9. The device as set forth in claim 1, wherein said therapeutic tool comprises a laser for tissue ablation.

10. The device as set forth in claim 1, wherein said elongate tubular member is a catheter, and wherein said catheter is for an intravascular or intracardiac medical procedure.

11. A method for guiding and delivering therapy inside of a body lumen, comprising:
(a) inserting an image-guided therapy device inside of said body lumen to an inner body region, wherein said image-guided therapy device comprises:
(i) an elongate tubular member having a distal end and an inner lumen;
(ii) an annular ultrasound array for real-time forward-looking acoustic imaging, wherein said annular ultrasound array is positioned on said distal end of said elongate tubular member, wherein said annulus of said annular ultrasound array defines a central lumen of said annular ultrasound array, wherein said central lumen of said annular ultrasound array and said inner lumen of said elongate tubular member are at least partially aligned, and wherein said annular ultrasound array comprises a plurality of capacitive micromachined ultrasonic transducers (cMUTs); and
(iii) a therapeutic tool, wherein said therapeutic tool is positioned near said distal end of said elongate tubular member and is positioned inside said inner lumen of said elongate tubular member;
(b) imaging at least a part of said inner body region by using said annular ultrasound array, wherein said imaging is in a forward direction;
(c) guiding said therapeutic tool to a focus spot based on said imaging; and
(d) applying therapy to said focus spot using said therapeutic tool.

12. The method as set forth in claim 11, wherein said cMUTs of said annular ultrasound array are operable at a high frequency, and wherein said high frequency is equal to or greater than about 10 MHz.

13. The method as set forth in claim 11, wherein said therapeutic tool comprises a high intensity focused ultrasound (HIFU) device, wherein said HIFU device is operable at a high frequency, and wherein said high frequency is equal to or greater than about 10 MHz.

14. The method as set forth in claim 13, wherein said HIFU device has a diameter of about 2 mm, wherein said HIFU device focuses ultrasonic energy to said focus spot, wherein said focus spot is located about 2 mm away from said HIFU device, and wherein the size of said focus spot is dependent on the frequency of said HIFU device.

15. The method as set forth in claim 13, wherein said HIFU device comprises a single focused transducer element.

16. The method as set forth in claim 13, wherein said HIFU device comprises a phased array of a plurality of HIFU cMUTs.

17. The method as set forth in claim 11, wherein said therapeutic tool comprises a laser for tissue ablation.

18. The method as set forth in claim 11, wherein said elongate tubular member is a catheter, and wherein said catheter is for an intravascular or intracardiac medical procedure.

19. The method as set forth in claim 11, further comprising scanning said image-guided therapy device over a scan area of said inner body region, wherein said scan area is larger than said focus spot, and wherein said scanning comprises imaging said scan area and applying therapy to at least a part of said scan area.

20. The method as set forth in claim 11, further comprising delivering an ultrasound contrast agent to enhance said imaging, wherein said ultrasound contrast agent is delivered through said inner lumen of said elongate tubular member.
